(19) United States
(12) Patent Application Publication
(10) Pub. No.: US 2023/0248858 A1
(43) Pub. Date: Aug. 10, 2023

(54) Dance Pole Ultraviolet Cleaning Device

(71) Applicant: Steven Capko, Weatherford, TX (US)

(72) Inventor: Steven Capko, Weatherford, TX (US)

(21) Appl. No.: 17/665,912

(22) Filed: Feb. 7, 2022

(51) Int. Cl. A61L 2/10; A47L 7/00; A47L 9/28; A47L 9/00; A63B 9/00

(52) U.S. Cl. CPC A61L 2/10; A47L 7/0004; A47L 7/0061; A47L 9/2857; A47L 9/009; A47L 9/2884; A47L 9/2805; A63B 9/00; A47L 2201/04; A61L 2202/17; A63B 2244/225

(57) ABSTRACT

The down and dirty sanitizer is the only product of its kind that is specifically designed to completely disinfect and sanitize dance poles, primarily used in adult entertainment, with an avant-garde UV light technology that works hand-in-hand with sound systems and a vacuum suctioning mechanism. The down and dirty sanitizer is uniquely designed with durable materials to ensure long-term sustainability and can be discreetly installed and operated in various settings where pole dancing is practiced and/or performed by varying dancers.

### BACKGROUND

Stripper poles in clubs and studios are often home to thousands of different strains of bacteria, germs, and pathogens, which can dramatically increase the likelihood of germ transmission between dancers who take turns utilizing the structure. Although some dancers opt to wipe the pole down after every set, the cleaning process can be incredibly time-consuming and the disinfectants that are used tend to leave a slippery residue on the pole's surface which could be a hazard, resulting in slips and falls during a performance. There have been no products available as original equipment or as an aftermarket to address this problem.

An apparatus to automatize the process of cleaning dance poles and like surfaces from bacteria, germs and pathogens without leaving a slippery residue on said surfaces is not being met by any known device or system at present. There have been no products available as original equipment or as an aftermarket to address this problem either.

### SUMMARY OF THE INVENTION

The main purpose of the dance pole ultraviolet cleaning device is to introduce a UV light sanitizing device intended for use on adult entertaining dance poles.

### BRIEF DESCRIPTION OF THE DRAWINGS

FIG. 1 is a top perspective view of the dance pole ultraviolet cleaning device in accordance with an embodiment of the present disclosure.

FIG. 2 is a top elevational view of the pole cleaning device in accordance with an embodiment of the present disclosure.

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

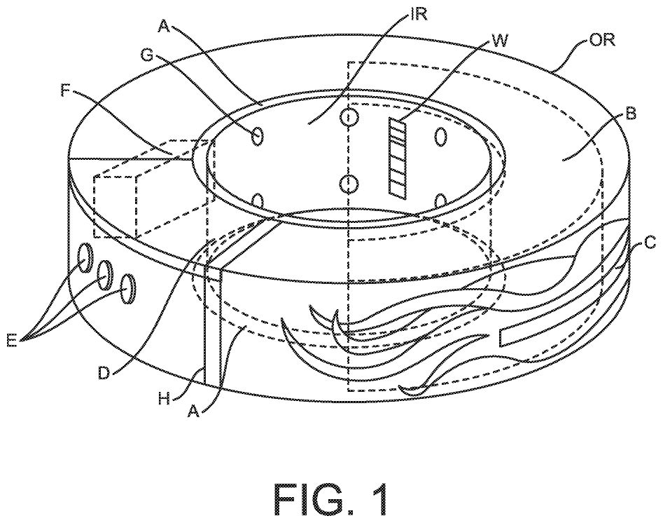

FIG. 1 is a top perspective view of the dance pole ultraviolet cleaning device showing: UV light referenced as A, motor referenced as B, running lights referenced as C, door for canister referenced as D, blinking lights referenced as E, rechargeable battery referenced as F, vacuum suction referenced as G, in accordance with an embodiment of the present disclosure. The view also includes the hinge H configured to open and close the device around a handrail or a pole as part of completed construction. The view also includes the wheels W the inside ring IR and the outside ring OR. Embodiments enable the device to travel up and down a pole by vacuum power and gravity alone. Embodiments also include wheel power to move the device up and down the pole against a gravity force thereon.

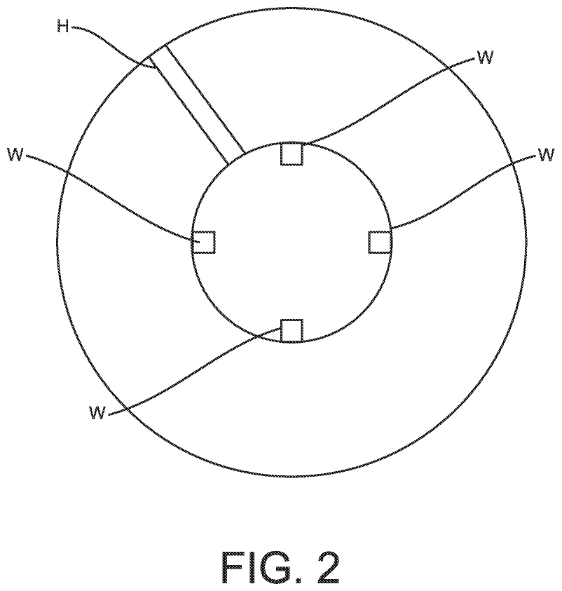

FIG. 2 is a top elevational view of the pole cleaning device in accordance with an embodiment of the present disclosure. The view includes the hinge H, the wheels W and the outer ring OR and the inner ring IR. The wheels W are seen protruding from the inner ring IR but are also countersunk in embodiments.

The present disclosed dance pole ultraviolet cleaning device, also known as the “Down And Dirty Sanitizer” or “D.A.D.S” offers a modem accessory that automatically sanitizes every inch of a dance pole, from top to bottom, after every performance with an ultraviolet light device. Formulated with pole care and cleaning in mind, the down and dirty sanitizer comes down from its ceiling, top of pole placement, and travels down the pole illuminating three hundred and sixty degree ultraviolet rays. The device will travel all the way from the docking station located on the ceiling to the floor as it thoroughly disinfects the dancing support medium. Once the device has successfully made its way down it will then travel back-up, killing 99.97% of germs approximately four times, based on its top, down and then return movement. To further enhance functionality, this innovative device is strategically programmed to connect to the primary sound equipment of a club which enables the device to sync with when songs start and end, often a cue to note the transition of a dancers set and thus an ideal time to start the cleaning. This cutting-edge device has the potential to transform and improve overall cleaning practices in clubs, studios, fireman poles, door handles escalator rails and other handrails, bus safety rails, shopping carts, and even gyms, as it thwarts the spread of dangerous germs between individuals to guarantee a safer and more sanitary pole dancing experience.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

What is claimed is:

1. A pole cleaning device comprising:
an annular housing having an inside ring defining holes and having a diameter larger than a diameter of the pole and having an outside ring;
an annular germicidal agent disposed around the inside ring and adapted to sterilize the pole around three hundred and sixty degrees; and
a vacuum motor configured to generate a suction from the inside ring holes onto the pole.

2. The pole cleaning device of claim 1, further comprising running lights disposed in the outside ring.

3. The pole cleaning device of claim 1, further comprising blinking lights disposed in the outside ring.

4. The pole cleaning device of claim 1, further comprising a compartment and a door thereto disposed in the outside ring for a maintenance thereof.

5. The pole cleaning device of claim 1, further comprising annular wheels configured to return the device to a beginning of the pole in a return cleaning cycle.

6. The pole cleaning device of claim 1, further comprising a rechargeable battery contained in the outside ring and configured to power the device.

7. The pole cleaning device of claim 1, further comprising annular magnets configured to attract the device to a metal handrail.

8. The pole cleaning device of claim 1, further comprising a hinge configured to enable an opening and a closing the device around the pole.

9. The pole cleaning device of claim 1, wherein the suction is configured to enable a drop of the device on the pole via a gravity force thereon.

10. The pole cleaning device of claim 1, wherein the annular germicidal agent is a UV light.

11. The pole cleaning device of claim 1, wherein the annular germicidal agent is a cleaning fluid disbursement device.

12. The pole cleaning device of claim 1, wherein the suction from the inside ring holes vacuum up a cleaning solution from the pole.
